(19) United States
(12) Patent Application Publication
(10) Pub. No.: US 2014/0296591 A1
(43) Pub. Date: Oct. 2, 2014

(54) METHOD FOR OPERATING HYDROPROCESSING SYSTEM

(71) Applicant: INTEVEP, S.A., Caracas (VE)

(72) Inventors: YTALO ORLANDO DAVILA GOMEZ, San Antonio de los Altos, M. Los Salias (VE); YLEYNA NILOHA ORELLANA ORTA, San Antonio de los Altos, M. Los Salias (VE)

(73) Assignee: Intevep, S.A., Caracas (VE)

(21) Appl. No.: 13/855,004

(22) Filed: Apr. 2, 2013

(51) Int. Cl. C07C 7/00

(52) U.S. Cl. CPC C07C 7/005
USPC 585/310

(57) ABSTRACT

Method for operating a two-stage hydroprocessing system includes introducing a feedstock and a first-stage gas to a first reaction zone a first pressure to generate a first-stage product stream; introducing the first-stage product stream to a first separation unit to separate an intermediate feedstock from a spent gas; introducing the intermediate feedstock and a second-stage gas to a second reaction zone a second pressure which is greater than the first pressure to generate a second-stage product stream; introducing the second-stage product stream to a second separation unit to separate a hydroprocessed product and an intermediate gas; feeding the intermediate gas directly to the first reaction zone as the first-stage gas; feeding spent gas to an amine unit and then compressor to generate recycled gas; and feeding recycled gas to the second reaction zone as the second-stage gas.

### BACKGROUND OF THE INVENTION

The invention relates to hydroprocessing of hydrocarbons, and more particularly to a modification of conventional hydroprocessing systems through alteration of the hydrogen-containing gas flow strategy.

Hydroprocessing and hydrotreatment systems are known wherein a feedstock is treated in a reactor in presence of a catalyst, along with a flow of hydrogen-containing gas. The hydrogen-containing gas reacts with the feedstock to provide an upgraded product. One common type of hydroprocess consists in removal of contaminants such as organic sulfur from the feedstock, generating H₂S. The resulting H₂S can then be removed from the treating gas, making possible to recycle back the treating gas to the reaction section.

In a conventional process, two or more stages of treatment in series may be used, and all stages are typically fed in parallel with required hydrogen from a single recycle compressor.

Such processes allow for upgrading of the hydrocarbon being treated, but the need exists for improved process efficiency, which is addressed by the present invention.

### SUMMARY OF THE INVENTION

In accordance with the present invention, a hydroprocessing method has been provided which combines reactor pressure strategy and hydrogen-containing gas flow strategy in order to: reduce compressor related investment and operating costs for new units and; revamp existing units to increase the treating gas to hydrocarbon ratio with minimal investment.

In accordance with the invention, a method for operating a two-stage hydroprocessing system comprising the steps of: introducing a feedstock and a first-stage hydrogen-containing gas to a first reaction zone operating at a first pressure to generate a first-stage product stream; introducing the first-stage product stream to a first separation unit to separate an intermediate feedstock and a spent hydrogen-containing gas; introducing the intermediate feedstock and a second-stage hydrogen-containing gas to a second reaction zone operating at a second pressure which is higher than the first pressure to generate a second-stage product stream; introducing the second-stage product stream to a second separation unit to separate a hydroprocessed product and an intermediate hydrogen-containing gas; feeding the intermediate hydrogen-containing gas directly to the first reaction zone as the first-stage hydrogen-containing gas; feeding the spent hydrogen-containing gas to an amine unit and then to a compressor to generate a recycle gas; and feeding the recycle gas to the second reaction zone as the second-stage hydrogen-containing gas. Make up of hydrogen-containing gas can be fed either at the suction or at the discharge of the compressor, or at the inlet of the first reaction zone together with the first-stage hydrogen-containing gas.

In further accordance with the invention, a method for upgrading operation of a conventional two-stage hydroprocessing system having a first reaction zone, a second reaction zone, and a compressor for feeding both the first reaction zone and the second reaction zone with hydrogen-containing gas in parallel at substantially the same operating pressure, comprises the steps of: feeding full capacity of hydrogen-containing gas from the compressor to the second reaction zone; separating hydrogen-containing gas from the second-stage product and feeding separated gas to the first reaction zone without need of additional compressor, due to differential pressure between the two reaction zones; separating hydrogen-containing gas from the first-stage product and feeding separated gas to an amine unit to remove contaminants and then to the compressor, wherein the second reaction zone is operated at a higher pressure than the first reaction zone.

By addressing the compressor needs of the system by feeding hydrogen-containing gas in a single serial flow first through the second reaction zone and then the first reaction zone and then back to the amine unit and compressor, substantial savings are accomplished by either significantly reducing the compressor capacity needed to properly pressurize the gas, or by increasing the actual treating gas to hydrocarbon ratio in each stage by feeding the entire quantity of hydrogen-containing gas from the compressor first through one reaction zone and then through the other.

In accordance with the invention, a minimal impact on the catalytic performance of the unit is achieved as compared to conventional two-stage hydroprocessing system, having a first reaction zone, a second reaction zone, and a compressor for feeding both the first reaction zone and the second reaction zone with hydrogen-containing gas in parallel at substantially the same operating pressure, since: hydrogen consumption in the second reaction zone is typically lower than that of the first reaction zone, and therefore the hydrogen-containing gas separated from second-stage product in the second separator has a higher hydrogen concentration and a similar flow-rate as compared to the hydrogen-containing gas separated from first-stage product in the first separator; operating pressure of first reaction zone and second reaction zone can be adjusted in order to have same average hydrogen partial pressure as compared to the conventional process.

In the case of reduced compressor size, this also provides savings to the operator of the method in terms of equipment and energy costs.

In the case of modification to an existing system, by continuing to use the original compressor, the ratio of hydrogen to feedstock in each reaction zone can be increased, which results in a larger catalyst lifecycle and a higher quality product for various reasons.

### BRIEF DESCRIPTION OF THE DRAWINGS

A detailed description of preferred embodiments of the present invention follows, with reference to the attached drawings, wherein:

FIG. 1 shows a conventional two-stage hydroprocessing system;

FIG. 2 shows a hydroprocessing system in accordance with the present invention;

FIG. 3 illustrates a conventional two-stage operating scheme utilized in a pilot scale test;

FIG. 4 illustrates a two stage operation scheme in accordance with the present invention utilized in a pilot scale test;

FIG. 5 illustrate results obtained from the pilot plants illustrated in FIGS. 3 and 4.

### DETAILED DESCRIPTION

The invention relates to a modification of a conventional hydroprocessing process which has two reaction stages with intermediate separation/stripping. The modification permits a reduction in operating and investment costs through an optimization of the treating gas use. Process integration and plant control are combined to potentially halve the inventory of recycle gas in a conventional process, without affecting the hydrogen/hydrocarbon ratio in the reactors. In this way, the investment cost in the recycle gas compressor and the associated amine system are reduced. Similarly, energy demand of the recycle gas compressor is reduced potentially by half.

This is accomplished by recirculation to the first reaction zone of the treating gas leaving the second reaction zone, through a pressure balance wherein operating pressure of the second reaction zone is greater than operating pressure of the first reaction zone. In a conventional process, both stages operate at the same pressure, and the hydrogen required for both reaction stages is handled by a single compressor of capacity sufficient for both stages combined. The present invention is based on realization that about 80% of the overall conversion is carried out in the first reaction step or stage, and this makes the content of contaminants in the treating gas leaving the second stage relatively low. Thus, according to the invention, gas from the second reaction zone can be directly recirculated to the first reaction zone without further purification.

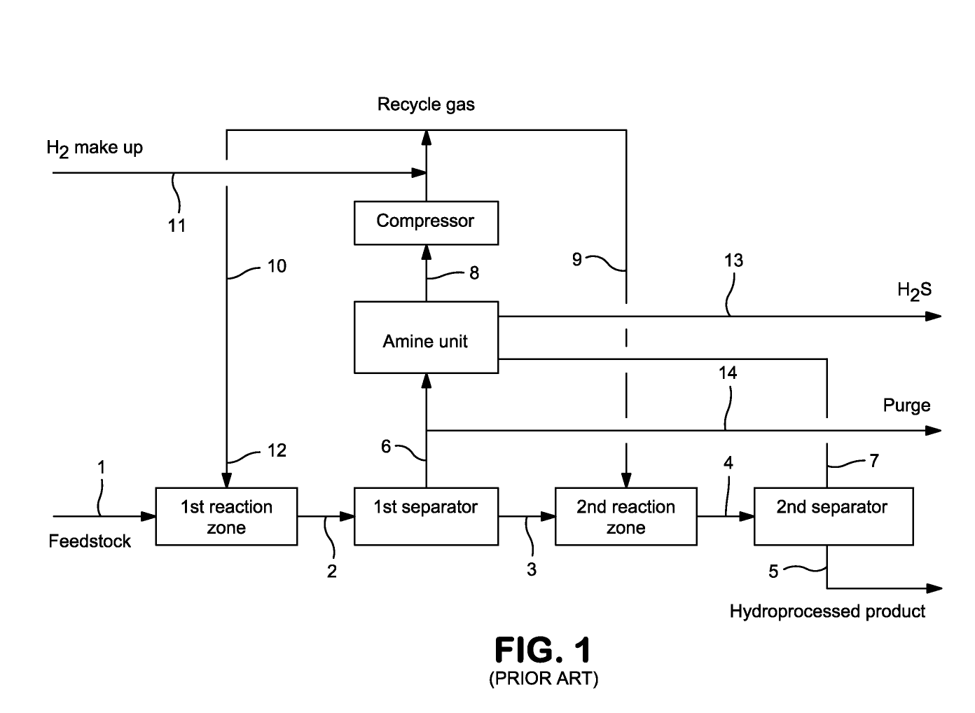

FIG. 1 illustrates a conventional process having a first reaction zone and a second reaction zone, having an intermediate first separator and a final second separator. Both, the intermediate first separator and the second separator can be comprised of one separator drum, a series of separator drums with intermediate cooling, and may or may not include an intermediate liquid product stripper.

As can be seen in FIG. 1, the flow path for feedstock is from incoming line 1 to first reaction zone, from first reaction zone to first separator (line 2), from first separator to second reaction zone (line 3), from second reaction zone to second separator (line 4), and the result is hydroprocessed product in line 5.

As shown in FIG. 1, the gas flow flows from first separator (line 6) and second separator (line 7) to an amine unit. As shown, at this location, H₂S and other contaminants are separated and removed in line 13, while the purified gas is sent to the compressor (line 8). Compressed gas is fed directly back to first reaction zone (line 10) and second reaction zone (line 9), with hydrogen make up (line 11) being added as needed. It should be clear from this process scheme that both, the amine unit and the compressor must have a capacity sufficient to purify, properly pressurize and recycle hydrogen-containing gas to feed both first reaction zone and second reaction zone. Purge gas line 14 can be taken from line 6 as shown in FIG. 1, or alternatively can be taken from line 7 or line 8, in order to keep a required hydrogen concentration in the recycle gas.

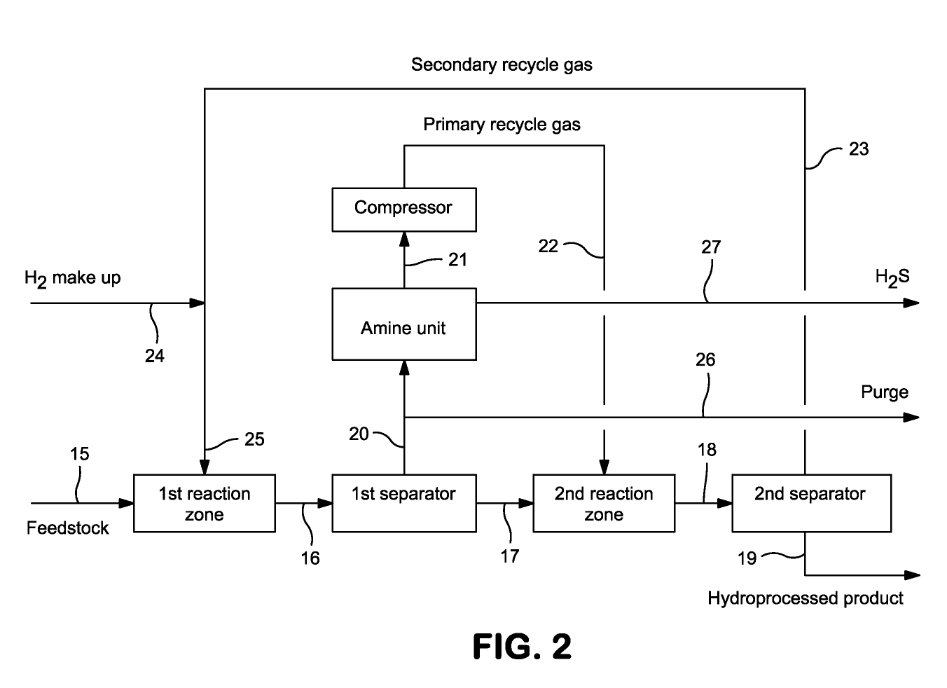

FIG. 2 illustrates a process in accordance with the present invention which also operates with a first reaction zone and a second reaction zone, and an intermediate separator between both reaction zones (first separator). A final separator identified as (second separator) is also present. Similarly to the conventional two-stage hydroprocessing system, the first separator can be comprised of one separator drum, a series of separator drums with intermediate cooling, and may also include an intermediate liquid product stripper. The second separator can be comprised of one separator drum or a series of separator drums with intermediate cooling.

Feedstock 15 in this process scheme follows a flow path similar to that of FIG. 1, and is passed through the first reaction zone. The first-stage product stream (line 16) is sent to the first separator where it is separated from the spent hydrogen-containing gas. The intermediate product (line 17) is then sent to the second reaction zone where the second-stage product (line 18) is generated. The second stage product is fed to the second separator where the hydroprocessed product (line 19) is recovered.

In accordance with the present invention, the gas flow is modified from the conventional scheme shown in FIG. 1. Specifically, gas flows from the first separator (line 20) to an amine unit where H₂S is removed. The hydrogen-containing gas leaving the amine unit (line 21) is sent to the compressor, and compressed primary recycle gas (line 22) is fed directly to the second reaction zone. Aside from whatever purge may be needed (line 26), the entire volume of gas that passes through the amine unit and compressor is fed to the second reaction zone. From second-stage product (line 18), hydrogen-containing gas is recovered in the second separator and fed through line 23 directly back to first reaction zone, without further compression or treatment, together with fresh hydrogen make up (line 24) through line 25. Thus, it should readily be apparent that the flow of gas in this configuration is serial, that is, it flows first through second reaction zone and then through first reaction zone, rather than in parallel as shown in FIG. 1 for conventional processes. As shown in FIG. 2, a hydrogen make up feed 24 can be incorporated as needed, preferably in the gas fed to the first reaction zone as shown. Alternatively, hydrogen make up can be fed at suction or at discharge of compressor. In order to keep hydrogen concentration in recycle gas to the required level, a purge line 26 can be directly taken from hydrogen-containing gas recovered in first separator. Alternatively, the purge gas line 26 can be taken either from compressor suction (line 21) or from compressor discharge (line 22).

It should be appreciated that hydrogen-containing gas flow in accordance with the method of the present invention essentially consists of, in sequence, flow through the second reaction zone, a separation unit, the first reaction zone, a separation unit, an amine unit, a compressor and back to the second reaction zone. The possibility of make up gas, purge and H₂S bleed as discussed in connection with FIG. 2 above are considered to fall within the essential scope of the above sequence. Thus, a sequence which includes some purge and/or H₂ make up is not considered to be outside the scope of the sequence discussed above.

It should also be noted that the separation units referred to above can be different separators, or a single separator with different separation zones, or any other modification of separator(s) which may be readily apparent to a person of ordinary skill in the art.

In accordance with the present invention, amine unit and compressor are preferably operated such that the gas is purified and pressurized to the operating pressure in second reaction zone which is higher than the operating pressure in first reaction zone. Operating pressure of second reaction zone can be controlled by manipulating the flow of hydrogen-containing gas in line 23 though a pressure control valve (not shown), while operating pressure of first reaction zone can be controlled by manipulating the flow of hydrogen-containing gas in line 20 through another pressure control valve (not shown). Purge line 26 and hydrogen make up line 24 are also manipulated to control the operating pressure of first reaction zone. Differential pressure between second reaction zone and first reaction zone is achieved by hydraulic losses through the system.

Operating conditions of each reaction zone include temperatures from 170° C. to 430° C., preferably from 200° C. to 390° C.; pressure from 200 psig to 2000 psig; liquid hourly space velocity from 0.3 to 10 h⁻¹. Operating pressure of first reaction zone is typically between 65 and 90% of operating pressure of second reaction zone. Each reaction zone can contain either one or a combination of several catalysts with the following functionalities: heteroatom removal, hydrogenation of unsaturated species or hydrocracking.

It should be appreciated that the pressure balance utilized to handle gas flow from amine unit and compressor to second reaction zone and then through second separator to first reaction zone results in the entire volume of hydrogen-containing gas being utilized in each reaction zone, and therefore provides an increase of the hydrogen to feedstock ratio in each reaction zone. In addition, a single flow through each reaction zone allows either a significant reduction in the size of the amine unit and compressor, or a significant increase in the hydrogen-containing gas which can be fed to each reactor. If the process is configured to reduce the size of the amine unit and compressor, the costs for this smaller capacity machinery would be reduced, as would the operating costs for energy and the like. If existing equipment is used, and instead the full flow of gas is passed to each reactor sequentially, the resulting larger ratio of hydrogen to feedstock can produce enhanced quality product through more complete removal of contaminant, potentially longer runs before total replacement of catalyst is needed, and the like.

As indicated above, the modification illustrated in FIG. 2 is made possible through the realization that approximately 65% to 85% of conversion is conducted in the first reaction zone. Thus, removing the impurities from the hydrogen-containing gas in first separator, typically H₂S and NH₃, prior to second reaction zone, significantly improves the performance of said second reaction zone. On the other hand, as conversion is approximately 15% to 35% in second reaction zone, the level of impurities in the hydrogen-containing gas is low enough, so it can be directly fed to the first reaction zone, without further purification, with minimal impact on its catalytic performance. With the proposed pressure balance and the hydrogen purity in the hydrogen-containing gas strategy, results are similar in overall performance, compared to the conventional two stage hydroprocessing scheme.

For a base case of a naphtha hydrotreatment unit of 35 MBPD of capacity, it is estimated that an investment cost reduction of US$8.5 MM can be obtained with respect to amine column and recycle gas compressor. Additionally, compression power is reduced by roughly 2000 hp, which translates into savings of around US$1.2 MM per year in operating costs (estimates as per 2010). Prior art approaches are based upon directioning of the recycle gas or the proportions in which it is distributed among the different reaction stages; however, they require multiple compressors to recover the operating pressure for each reaction stage, so there is no benefit in such systems on reduction of compressor capacity or reduction of capacity of the associated amine unit.

The present invention is not linked to a specific catalyst.

The present invention may or may not include a guard (diolefin saturation) reactor, upstream the first reaction stage.

Because of the lower gas flow, the diameter of the associate amine column is reduced, thus reducing the investment cost. There is no detriment to the hydrogen/hydrocarbon ratio in any reaction stage.

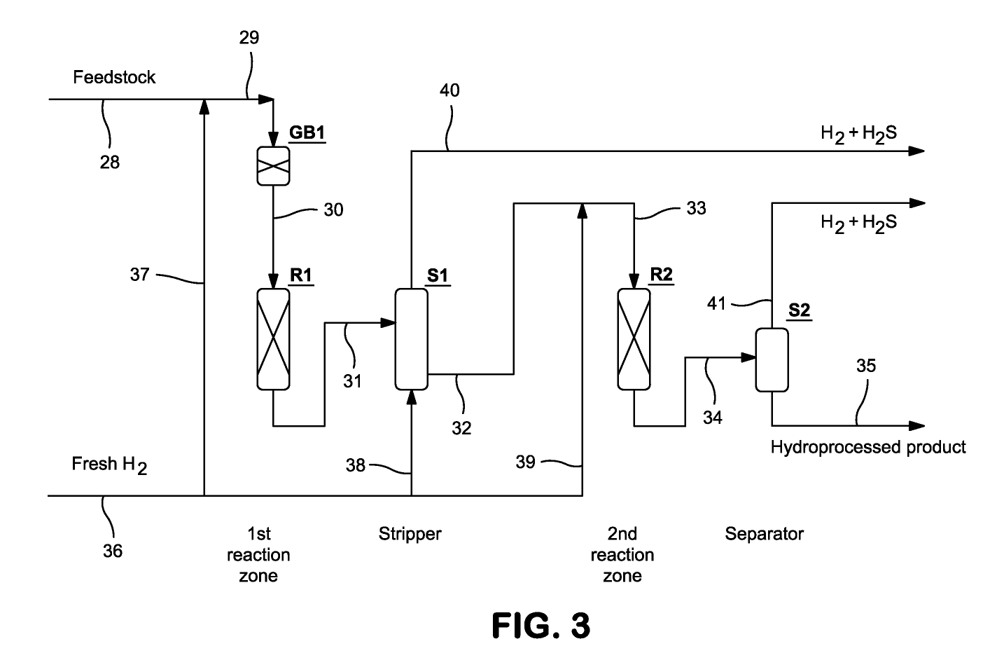

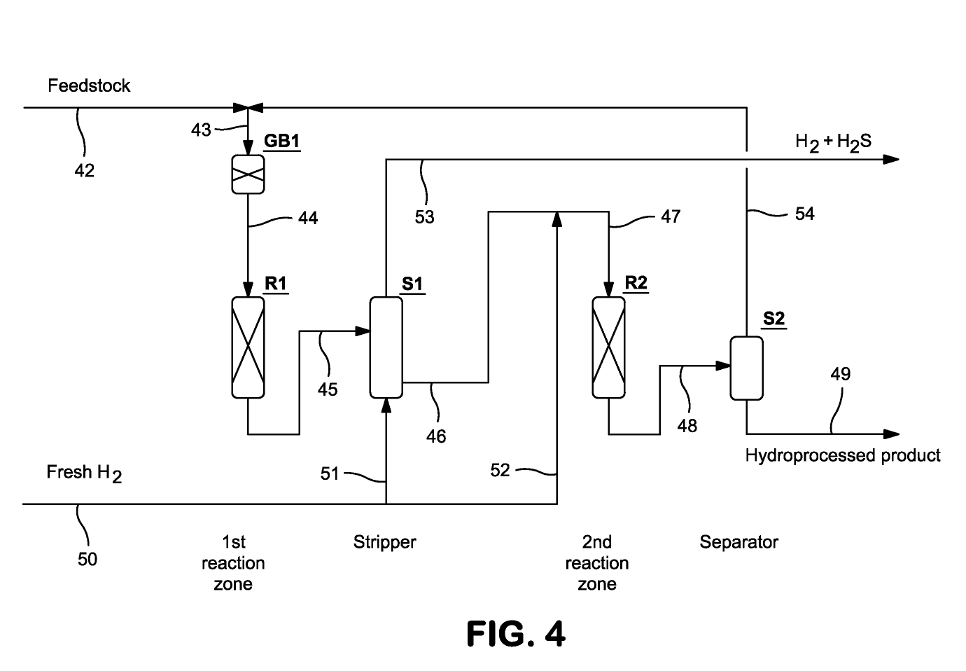

Pilot plant tests were performed in order to verify that both, the pressure balance as well as direct recirculation of hydrogen-containing gas from second reaction zone to first reaction zone do not effect the conversion or the selectivity. FIG. 3 depicts the arrangement used to emulate the conventional two-stage hydroprocess system, while FIG. 4 depicts the arrangement used to emulate the method according to the invention. The feedstock used in the test of these pilot plants is characterized as shown in Table 1.

TABLE 1

| Property | Value | Method |
|---|---|---|
| °API | 41.4 | ASTM D-1928 |
| Total sulfur (wt ppm) | 959 | ASTM D-5453-00 |
| Mercaptan sulfur (wt ppm) | 7.2 | UOP-163 |
| Bromine number | 27 | ASTM D-1159-01 |
| PIONA (% wt) | | ASTM D-6733 |
| Paraffins | 2.20 | — |
| Iso-paraffins | 13.11 | — |
| Olefins | 9.64 | — |
| Naphthens | 8.51 | — |
| Aromatics | 62.95 | — |
| C13+ | 2.94 | — |

Referring to FIG. 3, this system is based upon the prior art scheme of FIG. 1, and like reference numerals are used where appropriate.

As can be seen in FIG. 3, the flow path for feedstock is from incoming line 28 and mixed with fresh hydrogen-containing stream 37; the mixture then goes through line 29 to guard bed reactor GB1, where diolefins are selectively hydrogenated; then, the effluent of GB1 (stream 30) is fed to first reaction zone (R1), from first reaction zone to first separator (line 31) which at pilot scale is represented as a stripper (S1); liquid effluent from first separator (stream 32) is mixed with fresh hydrogen-containing gas (stream 39) and fed to second reaction zone (line 33); then from second reaction zone to second separator (line 34), which at pilot scale is represented as a flash drum separator (S2), and the result is hydroprocessed product in line 35.

As shown in FIG. 3, fresh hydrogen-containing gas is fed to the system through line 36, it is then injected in parallel to first reaction zone inlet (line 37), first separator (line 32) and second reaction zone (line 39); in the stripper (S1) H₂S and other contaminants are separated and removed at the top with a hydrogen-containing gas (line 40); similarly, in the second separator (S2) H₂S and other contaminants are separated and removed at the top with a hydrogen-containing gas (line 41). There is no amine unit for gas purification nor compressor for gas recycling. Injection of fresh hydrogen containing gas in line 36 emulates purification and compression of streams 40 and 41, which at commercial scale would be sent to an amine unit and a compressor, having a capacity sufficient to purify, properly pressurize and recycle hydrogen-containing gas to feed first reaction zone, second reaction zone and the stripper.

FIG. 4 shows a system based upon the process of the present invention as illustrated in FIG. 2, and like reference numerals are utilized where appropriate.

In FIG. 4, it can be seen that feedstock 42 follows a flow path similar to that of FIG. 2. It is mixed with secondary recycle gas (line 54) and is passed through the first reaction zone (line 43), which is composed of a guard bed reactor GB1 and first reactor R1. Product leaving GB1 is directly fed to R1 (line 44). Then, the first-stage product stream (line 45) is sent to the first separator (S1), which is represented at pilot scale as a stripper, where it is separated from the spent hydrogen-containing gas. The intermediate liquid product (line 46) is mixed with fresh hydrogen-containing gas (line 52) and then sent to the second reaction zone (R2) through line 47, where the second-stage product (line 48) is generated. The second stage product is fed to the second separator (S2), which is represented at pilot scale as a flash separator drum, where the hydroprocessed product (line 49) is recovered.

Similarly, the gas flow emulates the scheme presented in FIG. 2. Fresh hydrogen-containing gas is fed to the system through line 50, and is injected in parallel to the first separator (line 51) and inlet of second reaction zone (line 52). Injection of hydrogen-containing gas at the bottom of first separator is done to strip the first-stage product and remove H₂S and other contaminants, which are recovered at the top (line 53) together with the spent gas. Hydrogen-containing gas (line 52) is mixed with first-stage liquid product (line 46) and fed to second reaction zone (line 47). From second-stage product, hydrogen-containing gas is recovered in the second separator and fed through line 54 directly back to first reaction zone, without further compression or treatment. Thus, it should readily be apparent that the flow of gas in this configuration is serial, that is, it flows first through second reaction zone and then through first reaction zone, rather than in parallel. Second reaction stage operates at a higher pressure than first reaction stage. There are no amine purification nor compression stages, as injection of fresh high-purity hydrogen-containing gas (stream 50) emulates purification and compression of spent hydrogen-containing gas (line 53) as would happen at commercial scale. As no fresh hydrogen is directly injected to first reaction stage, at similar hydrogen to hydrocarbon ratio, significantly less fresh hydrogen-containing gas is fed to the system as compared to scheme shown in FIG. 3. In a commercial scale, this is translated into a lower capacity amine and compressor units.

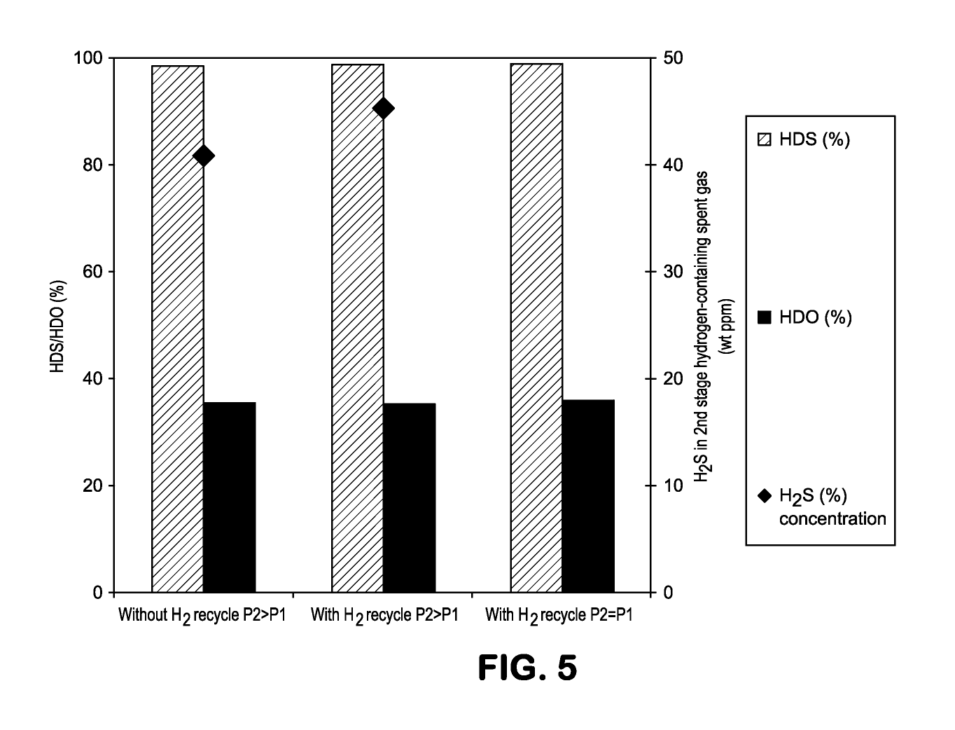

Results were monitored for the pilot plant operated in accordance with the schemes of FIGS. 3 and 4. In order to evaluate both the impact of second reaction stage spent hydrogen-containing gas direct recirculation to first reaction stage and the impact of pressure balance between second reaction zone and first reaction zone, three different experiments were carried out: first, direct recirculation of hydrogen-containing spent gas from second reaction zone (operating at 265° C. and 350 psig), recovered at second separator, to first reaction zone (operating at 275° C. and 250 psig), such that operating pressure of second reaction zone was higher than that of the first reaction zone (scheme of FIG. 4); second, without hydrogen-containing gas recirculation from second reaction zone (operating at 265° C. and 350 psig) to first reaction zone (operating at 275° C. and 250 psig), again with operating pressure of second reaction zone higher than that of the first reaction zone (scheme of FIG. 3); and third, with direct recirculation of hydrogen-containing spent gas from second reaction zone(operating at 265° C. and 290 psig), recovered at second separator, to first reaction zone (operating at 275° C. and 290 psig), such that operating pressure of second reaction zone was equal to that of the first reaction zone (scheme of FIG. 4). Overall desulfurization, overall olefins saturation and H₂S content in second reaction stage spent hydrogen-containing gas were monitored, and results are summarized in FIG. 5.

For example, FIG. 5 shows results in terms of overall hydrodesulphurization, which is substantially the same for all three studied cases. Similarly, olefins saturation is basically constant for all the studied cases. Therefore, it can be concluded that neither the pressure balance between both reaction zones nor the direct recirculation of untreated spent hydrogen-containing gas from second reaction zone to first reaction zone, have an impact on the unit performance, in terms of activity or selectivity. Similarly, H₂S content in second reaction stage spent gas is quite similar, demonstrating that conversion per stage is kept almost the same, having the unit the same overall performance.

FIG. 5 establishes that there is no significant difference in results obtained utilizing the flow scheme of the present invention as compared to a conventional process scheme, despite the fact that this flow scheme allows for utilization of a much smaller compressor and amine unit, and also a savings in energy for use in operating same.

It is to be understood that the invention is not limited to the illustrations described and shown herein, which are deemed to be merely illustrative of the best modes of carrying out the invention, and which are susceptible of modification of form, size, arrangement of parts and details of operation. The invention rather is intended to encompass all such modifications which are within its spirit and scope as defined by the claims.

1. A method for operating a two-stage hydroprocessing system, comprising the steps of:
introducing a feedstock and a first-stage hydrogen-containing gas to a first reaction zone operating at a first pressure to generate a first-stage product stream;
introducing the first-stage product stream to a separation unit to separate an intermediate feedstock and a spent hydrogen-containing gas;
introducing the intermediate feedstock and a second-stage hydrogen-containing gas to a second reaction zone operating at a second pressure which is higher than the first pressure to generate a second-stage product stream;
introducing the second-stage product stream to a separation unit to separate a hydroprocessed product and an intermediate hydrogen-containing gas;
feeding the intermediate hydrogen-containing gas directly to the first reaction zone as the first-stage hydrogen-containing gas;
feeding the spent hydrogen-containing gas to an amine unit and a compressor to generate a recycle gas; and
feeding the recycle gas to the second reaction zone as the second-stage hydrogen-containing gas.

2. The method of claim 1, wherein the first pressure and the second pressure are between about 200 psig and about 2000 psig.

3. The method of claim 2, wherein the first pressure is between about 65% and about 90% of the second pressure.

4. The method of claim 1, wherein gas flow of hydrogen-containing gas consists essentially of, in sequence, the second reaction zone, a separation unit, the first reaction zone, a separation unit, an amine unit, a compressor and back to the second reaction zone.

5. A method for upgrading operation of an existing two-stage hydroprocessing system having a first reaction zone, a second reaction zone, and a compressor for feeding both the first reaction and the second reaction zone with hydrogen-containing gas in parallel at substantially the same operating pressure, comprising the steps of:
feeding full capacity of hydrogen-containing gas from the compressor to the second reaction zone;
separating hydrogen-containing gas from the second reaction zone and feeding separated gas to the first reaction zone, wherein the second reaction zone is operated at a higher pressure than the first reaction zone;
separating hydrogen-containing gas from the first reaction zone and feeding separated gas to an amine unit to remove contaminants and then to the compressor.

6. The method of claim 5, wherein the first pressure and the second pressure are between about 200 psig and about 2000 psig.

7. The method of claim 6, wherein the first pressure is between about 65% and about 90% of the second pressure.

8. The method of claim 5, wherein gas flow of hydrogen-containing gas consists essentially of, in sequence, the second reaction zone, a separation unit, the first reaction zone, a separation unit, an amine unit, a compressor and back to the second reaction zone.

9. The method of claim 5, wherein a ratio of hydrogen to hydrocarbon is higher in the upgraded operation than in the existing system.
